(19) United States
(12) Patent Application Publication
(10) Pub. No.: US 2021/0025001 A1
(43) Pub. Date: Jan. 28, 2021

(54) Methods for Detecting and Treating Idiopathic Pulmonary Fibrosis

(71) Applicant: THE TRANSLATIONAL GENOMICS RESEARCH INSTITUTE, Phoenix, AZ (US)

(72) Inventors: Matthew J. Huentelman, Phoenix, AZ (US); Ignazio Piras, Phoenix, AZ (US)

(73) Assignee: THE TRANSLATIONAL GENOMICS RESEARCH INSTITUTE, Phoenix, AZ (US)

(21) Appl. No.: 16/938,847

(22) Filed: Jul. 24, 2020

Related U.S. Application Data
(60) Provisional application No. 62/878,294, filed on Jul. 24, 2019.

(51) Int. Cl. C12Q 1/6883; A61K 31/496; A61K 31/4418; A61K 31/573; A61K 31/5355; A61K 31/365; A61K 31/52; A61K 39/395; A61K 31/4439; A61K 31/675

(52) U.S. Cl. CPC C12Q 1/6883; A61K 31/496; A61K 31/4418; A61K 31/573; A61K 31/5355; A61K 31/365; C12Q 2600/124; A61K 39/3955; A61K 31/4439; A61K 31/675; C12Q 2600/106; C12Q 2600/156; A61K 31/52

(57) ABSTRACT

Methods are provided for diagnosing and treating idiopathic pulmonary fibrosis (IPF) in humans and canine idiopathic pulmonary fibrosis (CIPF) in canines. The methods include detecting expression of genes found to indicate a predisposition, a risk, or a presence of IPF: SDHAF2, CPSF7, and MUC5B. One variant, rs22669389, corresponding to position 54992254 on canine (CanFam3.1) chromosome 18, was identified at a suggestive level of significance to be associated with CIPF. The methods further comprise performing whole genome sequencing (WGS) of DNA in the sample to confirm detection of a variant indicating a predisposition, a risk, or a diagnosis of IPF or CIPF. The method further includes treating a subject for IPF or CIPF, based on the diagnosis of IPF or CIPF.

### CROSS REFERENCE TO RELATED APPLICATIONS

The present application claims priority to U.S. Provisional Application Ser. No. 62/878,294 filed on Jul. 24, 2019, the contents of which is hereby incorporated by reference in its entirety.

### FIELD

The present invention relates to the field of methods of diagnosing and treating lung disease, and more specifically, to diagnosing and treating Idiopathic Pulmonary Fibrosis.

### BACKGROUND

Canine idiopathic pulmonary fibrosis (CIPF) is a chronic and progressive fibrotic lung disease that particularly affects the West Highland White Terrier (WHWT) dog breed [1]. CIPF shares several clinical and pathological features with human IPF and it has been proposed as a possible sporadic disease model [2]. A typical clinical feature occurring in the majority of affected dogs is inspiratory crackles, as well as laryngo-tracheal reflex, tachypnea, and excessive abdominal breathing [2,3]. Human IPF is considered to be a disease of the epithelium. Specifically, microscopic injuries of the aging lung epithelium lead to defective regeneration and abnormal epithelial-mesenchymal crosstalk with activation of transforming growth factor beta (TGF-β) [4,5]. This is followed by extravascular coagulation, immune system activation, and secretion of excessive amounts of extracellular matrix (ECM) proteins [2]. However, the overall initiating cause of this pathological cascade in dogs and humans is still unknown.

Several studies have attempted to clarify the molecular mechanisms of CIPF in WHWTs. Upregulation of activin A has been reported in the lung alveolar epitelium of WHWTs with CIPF [6]. Furthermore, increased TGF-β1 signaling activity was detected in WHWTs and other predisposed breeds (such as Scottish Terriers and Bichons Frise) compared to non-predisposed breeds [7]. In an RNA expression profiling study in dogs, chemokine and interleukin genes were found to be overexpressed in the lungs, with CCL2 mRNA levels also noted as being elevated in serum [8]. Endothelin-1, measured in both serum and bronchoalveolar lavage fluid, has been suggested to be a biomarker suitable to differentiate dogs with CIPF from dogs with chronic bronchitis and eosinophilic bronchopneumopathy [9].

Genetic background is considered one of the risk factors for both CIPF and IPF [3,10]. Genome-wide association studies (GWAS) have led to the detection of several genes linked with the disease in humans. Specifically, three GWAS have been conducted in humans detecting signals in AKAP13; MUC5B; DSP; TOLLIP; MDGA2; SPPL2C; and TERT [10,11,12]. However, genetic risk factors for CIPF in the WHWT have not been identified. The domestic dog (Canis familiaris) is a useful model for many human diseases [13] due to the high number of analogous diseases [14], similar physiologies and medical care, as well as the simplified genetic architecture in purebred dogs [15]. Each dog breed originated from a small founder population with consequently low levels of genomic heterogeneity and long stretches of linkage disequilibrium (LD). Due to these characteristics, GWAS in dogs have increased statistical power comparable to, or better than, those performed in human population isolates [16]. This genetic homogeneity, the consequence of strong artificial selection conducted by humans, also led to an excess of inherited diseases, offering unique opportunities to discover genetic associations for spontaneous diseases [13]. Several GWAS have been conducted in specific breeds using the canine single nucleotide polymorphisms (SNP) array leading to the discovery of genetic risk factors for ectopic ureters [17], inflammatory bowel disease [18], hereditary ataxia [19], and hypothyroidism [20], among others.

### SUMMARY

A need exists for methods for detecting and treating idiopathic pulmonary fibrosis (IPF) in humans and canine idiopathic pulmonary fibrosis (CIPF) in dogs. CIPF may have molecular pathological overlap with human lung fibrotic disease. An evaluation of canine IPF, a disorder having clinical similarities to IPF in humans, is needed for identifying at risk and affected dogs, allowing development of targeted therapy and identifying similar genetic factors that are associated with the development of IPF in humans.

Variants are identified herein that indicate a predisposition, a risk, or a presence of IPF or CIPF. A method for treating a subject for IPF is disclosed. The method may comprise the steps of extracting genomic DNA from a sample from the subject, assaying the genomic DNA for one or more single nucleotide polymorphisms (SNPs), and detecting at least one of a T allele at position 54992254 on canine (CanFam3.1) chromosome 18, a C allele at position 54987884 on canine (CanFam3.1) chromosome 18, an A allele at position 54986491 on canine (CanFam3.1) chromosome 18, an A allele at position 54986070 on canine (CanFam3.1) chromosome 18, an A allele at position 54992285 on canine (CanFam3.1) chromosome 18, a G allele at position 54987464 on canine (CanFam3.1) chromosome 18, an A allele at position 54983627 on canine (CanFam3.1) chromosome 18, a G allele at position 54984004 on canine (CanFam3.1) chromosome 18, a C allele at position 54987912 on canine (CanFam3.1) chromosome 18, and a C allele at position 54986170 on canine (CanFam3.1) chromosome 18. The method may further include administering an effective amount of a treatment to the subject. The treatment may include a brohchodilator, a steroid, an anti-fibrotic drug, an anti-inflammatory drug, Nintedanib, Pirfenidone, prednisone, Mycophenolate mofetil, mycophenolic acid, Azathioprine, pamrevlumab, omeprazole, cyclophosphamide, oxygen therapy, pulmonary rehabilitation, or organ transplantation.

In another embodiment, the method further comprises performing whole genome sequencing (WGS) of DNA in the sample to confirm detection of a variant indicating a predisposition for, a risk of, or a diagnosis of IPF or CIPF.

According to various embodiments, methods are disclosed for diagnosing and treating a subject for IPF. The method may comprise the steps of extracting genomic DNA from a sample derived from the subject, determining in the subject-derived sample an expression of a gene selected from the group consisting of succinate dehydrogenase complex assembly factor 2 (SDHAF2), cleavage and polyadenylation specific factor 7 (CPSF7), and mucin 5B, oligomeric mucus/gel-forming (MUC5B), and diagnosing the subject as having IPF based on the expression of the gene being different than a normal control sample or based on the expression meeting a threshold level. The method may further include the step of administering to the subject diagnosed as having IPF an effective amount of a pharmaceutical composition selected from the group consisting of a brohchodilator, a steroid, an anti-fibrotic composition, and an anti-inflammatory composition.

In further embodiments, methods are provided for breeding a canine subject to reduce propensity to CIPF in progeny resulting from the breeding. The method may include the steps of extracting genomic DNA from a sample from the canine subject, assaying the genomic DNA for one or more single nucleotide polymorphisms (SNPs), detecting the SNPs corresponding to at least one of: position 54992254 on canine (CanFam3.1) chromosome 18, position 54987884 on canine (CanFam3.1) chromosome 18, position 54986491 on canine (CanFam3.1) chromosome 18, position 54986070 on canine (CanFam3.1) chromosome 18, position 54992285 on canine (CanFam3.1) chromosome 18, position 54987464 on canine (CanFam3.1) chromosome 18, position 54983627 on canine (CanFam3.1) chromosome 18, position 54984004 on canine (CanFam3.1) chromosome 18, position 54987912 on canine (CanFam3.1) chromosome 18, and position 54986170 on canine (CanFam3.1) chromosome 18. The method may further include breeding the canine subject with at least one of: an A allele at position 54992254 on canine (CanFam3.1) chromosome 18, a T allele at position 54987884 on canine (CanFam3.1) chromosome 18, a G allele at position 54986491 on canine (CanFam3.1) chromosome 18, a G allele at position 54986070 on canine (CanFam3.1) chromosome 18, a G allele at position 54992285 on canine (CanFam3.1) chromosome 18, a T allele at position 54987464 on canine (CanFam3.1) chromosome 18, a G allele at position 54983627 on canine (CanFam3.1) chromosome 18, an A allele at position 54984004 on canine (CanFam3.1) chromosome 18, a T allele at position 54987912 on canine (CanFam3.1) chromosome 18, and a T allele at position 54986170 on canine (CanFam3.1) chromosome 18.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the figures, wherein like numerals may denote like elements.

FIGS. 1A and 1B illustrate single nucleotide polymorphism (SNP)-level analysis and genome level analysis of the detected variants using Manhattan plots;

FIGS. 2A and 2B illustrate plots showing the details of the association region in chromosome 18 on canine reference genome CanFam3.1; and

FIG. 3 illustrates a distribution of the pi-hat value computed between each pair of dogs included in the genome-wide association study (GWAS).

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

In the present disclosure, a genome-wide association study (GWAS) in the WHWT was conducted using whole genome sequencing (WGS) to discover genetic variants associated with CIPF with a goal of finding genetic risk factors that may predispose the WHWT to the disease. This study identified significant signals, or markers, at the gene level in both the cleavage and polyadenylation specific factor 7 (CPSF7) and succinate dehydrogenase complex assembly factor 2 (SDHAF2) genes (adjusted p=0.016 and 0.024, respectively), which are two overlapping genes located on canine chromosome 18. CPSF7 is associated with lung adenocarcinoma, further highlighting the potential relevance of these disclosed results because IPF and lung cancer share several pathological mechanisms.

As used herein, “amplification reaction” refers to a method of detecting target nucleic acid by in vitro amplification of DNA or RNA.

As used herein, “polymerase chain reaction (PCR)” refers to the amplification of a specific DNA sequence, termed target or template sequence, that is present in a mixture, by adding two or more short oligonucleotides, also called primers, that are specific for the terminal or outer limits of the template sequence. The template-primers mixture is subjected to repeated cycles of heating to separate (melt) the double-stranded DNA and cooling in the presence of nucleotides and DNA polymerase such that the template sequence is copied at each cycle.

The term “primer” refers to DNA oligonucleotides complementary to a region of DNA and serves as the initiation of amplification reaction from the 5′ to 3′ direction. For example, a forward and a reverse marker-specific primer can be designed to amplify the marker from a nucleic acid sample.

The term “primer pair” refers to the forward and reverse primers in an amplification reaction leading to amplification of a double-stranded DNA region of the target.

The term “target” or “marker” refers to a nucleic acid region bound by a primer pair that is amplified through an amplification reaction. The PCR “product” or “amplicon” is the amplified nucleic acid resulting from PCR of a set of primer pairs. In some embodiments, the term “marker” encompasses a gene and a gene allele thereof, and the products (i.e., RNA) of the gene or a gene allele thereof, whose expression or activity is directly or indirectly associated with a particular phenotype or cellular condition, or physiological characteristic.

An allele includes any form of a particular nucleic acid that may be recognized as a particular form on account of its location, sequence, chemical modification of the sequence, expression level, expression specificity or any other characteristic that may identify it as being a form of the particular gene. Variable alleles of a particular gene may differ from each other because of point mutations, silent mutations, deletions, insertions, frameshift mutations, single nucleotide polymorphisms (SNPs), inversions, translocations, heterochromatic insertions, differentially epigenetically modified, or any combination of thereof, relative to a reference gene. Different alleles may, but need not, result in detectable differences in gene expression or protein functions. An allele of a gene may or may not encode proteins or peptides. Different alleles may differ in expression level, pattern, temporal or spatial specificity, and expression regulation. In the case of encoded proteins, the protein from different alleles may or may not be functional. Further, the protein may be gain-of-function, loss-of-function, or with altered function. An allele may be compared to another allele that may be termed a wild type form of an allele. In comparison to the wild type allele, a different allele may be called a mutation or a mutant. Mutants may also be interchangeably called variants. In some cases, the wild type allele is more common than the mutant. In the example of gene mutation, the mutation may be in the coding region or the non-coding region. The non-coding region comprises transcriptional and translational control elements. Suitable transcription or translation control elements include but are not limited to upstream control elements, enhancer elements, TATA boxes, cis regulatory regions, activator binding regions, repressor binding regions, transcription initiation sites, polyadenylation control elements, transcription termination sites, ribosome binding sites, translation initiation sites, and translation termination sites.

A haplotype may be any combination of one or more closely linked alleles inherited as a unit with little genetic shuffling across generations. The genetic sequences of different individuals are remarkably similar. When the chromosomes of two humans are compared, their DNA sequences can be identical for hundreds of bases. But at about one in every 1000 to 1,200 bases, on average, the sequences will differ. As such, one individual might have an A at that location, while another individual has a G, or a person might have extra bases at a given location or a missing segment of DNA. Differences in individual bases are the most common type of genetic variation. These genetic differences are known as single nucleotide polymorphisms (SNPs). Given the relatively close spacing between some SNPs, SNPs are typically inherited in blocks.

“Linked”, “linkage”, or “allelic association” means the preferential association of a particular allele or genetic marker with a specific allele or genetic marker at a nearby chromosomal location more frequently than expected by chance for any particular allele frequency in the population. For example, if locus X has alleles “a” and “b,” which occur equally frequently, and linked locus Y has alleles “c” and “d,” which occur equally frequently, one would expect the combination “ac” to occur with a frequency of 0.25. If “ac” occurs more frequently, then alleles “a” and “c” are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable.

An allele of a gene may have overexpression, underexpression or no expression. Alternatively, an allele of a gene may or may not produce a functional protein. A gene allele may produce a protein with altered sequence, function, localization, stability, dimerization, protein-protein interaction, or temporal or spacial expression specificity. A genetic mutation or variance may be any detectable change in genetic material such as DNA, or a corresponding change in the RNA or protein product of that genetic material. The presence or absence of an allele may be detected through the use of any process through which a specific nucleic acid molecule may be detected, including direct and indirect methods of detecting the presence or absence of the specific nucleic acid.

“Amplification” is a special case of nucleic acid replication involving template specificity. Amplification may be a template-specific replication or a non-template-specific replication (i.e., replication may be specific template-dependent or not). Template specificity is here distinguished from fidelity of replication (synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out. The amplification process may result in the production of one or more amplicons.

The term “template” refers to nucleic acid originating from a sample that is analyzed for the presence of one or more markers. In contrast, “background template” or “control” is used in reference to nucleic acid other than sample template that may or may not be present in a sample. Background template is most often inadvertent. It may be the result of carryover, or it may be due to the presence of nucleic acid contaminants sought to be purified out of the sample. For example, nucleic acids from organisms other than those to be detected may be present as background in a test sample.

The term “amplifiable nucleic acid” refers to nucleic acids that may be amplified by any amplification method. It is contemplated that “amplifiable nucleic acid” will usually comprise “sample template.” The terms “PCR product,” “PCR fragment,” “amplification product,” and “amplicon” refer to the resultant mixture of compounds after two or more cycles of the PCR steps of denaturation, annealing and extension. These terms encompass the case where there has been amplification of one or more segments of one or more target sequences.

Detection according to some embodiments of the disclosure may comprise contacting the amplified nucleic acid with a probe; and detecting the hybridization of probe with the amplified nucleic acid. Detection may be performed by a variety of methods, such as but not limited to, by a nucleic acid amplification reaction. In some embodiments the amplification reaction may be an end-point determination or the amplification reaction may be quantitative. The quantification may be a real-time PCR method. In some embodiments, the real-time PCR may be a SYBR® Green Assay or a TAQMAN® Assay. Detection, in various embodiments, may be performed by hybridization using probes specific to target sequences. According to various embodiments, combinations of amplification and hybridization may be used for detection.

As used herein, “real-time PCR” may refer to the detection and quantitation of a DNA or a surrogate thereof in a sample.

Generally, some embodiments of the present invention can be used to detect, identify, assess, sequence, or otherwise evaluate a target or marker. A marker may be any molecular structure produced by a cell, expressed inside the cell, accessible on the cell surface, or secreted by the cell. A marker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. A marker may also be called a target and the terms are used interchangeably.

A marker may be represented by the sequence of a nucleic acid from which it can be derived or any other chemical structure. Examples of such nucleic acids include miRNA, tRNA, siRNA, mRNA, cDNA, or genomic DNA sequences including complimentary sequences. Alternatively, a marker may be represented by a protein sequence. The concept of a marker is not limited to the products of the exact nucleic acid sequence or protein sequence by which it may be represented. Rather, a marker encompasses all molecules that may be detected by a method of assessing the expression of the marker. Examples of molecules encompassed by a marker include point mutations, silent mutations, insertions or deletions (INDELS), frameshift mutations, translocations, alternative splicing derivatives, differentially methylated sequences, differentially modified protein sequences, truncations, soluble forms of cell membrane associated targets, and any other variation that results in a product that may be identified as the target.

Expression encompasses any and all processes through which material derived from a nucleic acid template may be produced. Expression thus includes processes such as RNA transcription, mRNA splicing, protein translation, protein folding, post-translational modification, membrane transport, associations with other molecules, addition of carbohydrate moeties to proteins, phosphorylation, protein complex formation and any other process along a continuum that results in biological material derived from genetic material whether in vitro, in vivo, or ex vivo. Expression also encompasses all processes through which the production of material derived from a nucleic acid template may be actively or passively suppressed. Such processes include all aspects of transcriptional and translational regulation. Examples include heterochromatic silencing, transcription factor inhibition, any form of RNAi silencing, microRNA silencing, alternative splicing, protease digestion, posttranslational modification, and alternative protein folding.

Expression may be assessed by any number of methods used to detect material derived from a nucleic acid template used currently in the art and yet to be developed. Examples of such methods include any nucleic acid detection method including the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, direct sequencing of genomic DNA, or any other method of detecting a specific nucleic acid now known or yet to be disclosed. Other examples include any process of assessing protein expression including flow cytometry, immunohistochemistry, ELISA, Western blot, and immunoaffinity chromatograpy, HPLC, mass spectrometry, protein microarray analysis, PAGE analysis, isoelectric focusing, 2-D gel electrophoresis, or any enzymatic assay.

Differential expression encompasses any detectable difference between the expression of a marker in one sample relative to the expression of the marker in another sample. Differential expression may be assessed by a detector, an instrument containing a detector, or by aided or unaided human eye. Examples include but are not limited to differential staining of cells in an assay configured to detect a marker, differential detection of bound RNA on a microarray to which a sequence capable of binding to the marker is bound, differential results in measuring RT-PCR measured in Ct or alternatively in the number of PCR cycles necessary to reach a particular optical density at a wavelength at which a double stranded DNA binding dye (e.g. SYBR Green) incorporates, differential results in measuring label from a reporter probe used in a real-time RT-PCR reaction, differential detection of fluorescence on cells using a flow cytometer, differential intensities of bands in a Northern blot, differential intensities of bands in an RNAse protection assay, differential cell death measured by apoptotic markers, differential cell death measured by shrinkage of a tumor, or any method that allows a detection of a difference in signal between one sample or set of samples and another sample or set of samples.

Some embodiments of the invention may include a method of comparing a marker in a sample relative to one or more control samples. The expression of the marker in a sample may be compared to a level of expression predetermined to predict the presence or absence of a particular physiological characteristic. The level of expression may be derived from a single control or a set of controls. A control may be any sample with a previously determined level of expression. A control may comprise material within the sample or material from sources other than the sample. Alternatively, the expression of a marker in a sample may be compared to a control that has a level of expression predetermined to signal or not signal a cellular or physiological characteristic. This level of expression may be derived from a single source of material including the sample itself or from a set of sources. Comparison of the expression of the marker in the sample to a particular level of expression results in a prediction that the sample exhibits or does not exhibit the cellular or physiological characteristic.

The disclosure contemplates assessing the expression of the marker in any biological sample from which the expression may be assessed. One skilled in the art would know to select a particular biological sample and how to collect said sample depending upon the marker that is being assessed. Examples of sources of samples include but are not limited to biopsy or other in vivo or ex vivo analysis of prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus. In some aspects of the disclosure, the sample comprises a fluid sample, such as peripheral blood, lymph fluid, ascites, serous fluid, pleural effusion, sputum, cerebrospinal fluid, amniotic fluid, lacrimal fluid, saliva, stool, or urine. Samples include single cells, whole organs or any fraction of a whole organ, in any condition including in vitro, ex vivo, in vivo, post-mortem, fresh, fixed, or frozen.

Additionally, a sample may be derived from a subject, such as a plant or animal, including humans. As used herein, the term “subject” or “patient” refers to any organism subject or susceptible to IPF including mammals, further including humans. For example, subject may refer to a human or a non-human animal. In some aspects, subject refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In further embodiments, the subject is a human. In some embodiments, the subject is an animal, or more specifically, a canine. The animal may be a canine such as a Pug Dog, Chihuahua, West Highland White Terrier, Pekingese, Labrador retriever, Golden retriever, Beagle, German shepherd, Dachshund, Yorkshire terrier, Scottish Terrier, Boxer, Poodle, Shih tzu, Miniature schnauzer, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other breeds.

Prediction of a cellular or physiological characteristic includes the prediction of any cellular or physiological state that may be predicted by assessing the expression of a marker. Examples include but are not limited to the likelihood that one or more diseases is present or absent, the likelihood that a present disease will progress, remain unchanged, or regress, the degree to which a disease will respond or not respond to a particular therapy. Further examples include the likelihood that a cell will move, senesce, apoptose, differentiate, metastasize, or change from any state to any other state or maintain its current state.

Expression of a marker in a sample may be more or less than that of a level predetermined to predict the presence or absence of a cellular or physiological characteristic. The expression of the marker in the sample may be more than 1,000,000×, more than 100,000×, more than 10,000×, more than 1000×, more than 100×, more than 10×, more than 5×, more than 2×, about 1×, more than 0.5×, more than 0.1× more than 0.01×, more than 0.001×, more than 0.0001×, more than 0.00001×, more than 0.000001×, more than 0.0000001× or less than 0.0000001× that of a level predetermined to predict the presence or absence of a cellular or physiological characteristic.

One type of cellular or physiological characteristic is the risk that a particular disease outcome will occur. Assessing this risk includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

Determining the level of expression that signifies a physiological or cellular characteristic may be assessed by any of a number of methods. The skilled artisan will understand that numerous methods may be used to select a level of expression for a particular marker or a plurality of markers that signifies a particular physiological or cellular characteristic. In diagnosing the presence of a disease, a threshold value may be obtained by performing the assay method on samples obtained from a population of patients having a certain type of disease (fungal infection for example,) and from a second population of subjects that do not have the disease. In assessing disease outcome or the effect of treatment, a population of patients, all of which have, a disease such as a fungal infection, may be followed for a period of time. After the period of time expires, the population may be divided into two or more groups. For example, the population may be divided into a first group of patients whose disease progresses to a particular endpoint and a second group of patients whose disease does not progress to the particular endpoint. Examples of endpoints include disease recurrence, death, metastasis or other states to which disease may progress. If expression of the marker in a sample is more similar to the predetermined expression of the marker in one group relative to the other group, the sample may be assigned a risk of having the same outcome as the patient group to which it is more similar.

In addition, one or more levels of expression of the marker may be selected that provide an acceptable ability of its ability to signify a particular physiological or cellular characteristic. Examples of such characteristics include identifying or diagnosing a particular disease, assessing a risk of outcome or a prognostic risk, or assessing the risk that a particular treatment will or will not be effective.

Some embodiments of the invention may comprise the use of one or more methods of amplifying a nucleic acid-based starting material (i.e., a template, including genomic DNA, crude DNA extract, single-stranded DNA, double-stranded DNA, cDNA, RNA, or any other single-stranded or double-stranded nucleic acids). Nucleic acids may be selectively and specifically amplified from a template nucleic acid contained in a sample. In some nucleic acid amplification methods, the copies are generated exponentially. Examples of nucleic acid amplification methods known in the art include: polymerase chain reaction (PCR), ligase chain reaction (LCR), self-sustained sequence replication (3SR), nucleic acid sequence based amplification (NASBA), strand displacement amplification (SDA), amplification with Qβ replicase, whole genome amplification with enzymes such as φ29, whole genome PCR, in vitro transcription with T7 RNA polymerase or any other RNA polymerase, or any other method by which copies of a desired sequence are generated.

In addition to genomic DNA, any polynucleotide sequence can be amplified with an appropriate set of primer molecules. In particular, the amplified segments created by the PCR process itself are, themselves, efficient templates for subsequent PCR amplifications.

PCR generally involves the mixing of a nucleic acid sample, two or more primers or oligonucleotides (primers and oligonucleotides are used interchangeably herein) that are designed to recognize the template DNA, a DNA polymerase, which may be a thermostable DNA polymerase such as Taq or Pfu, and deoxyribose nucleoside triphosphates (dNTP's). In some embodiments, the DNA polymerase used can comprise a high fidelity Taq polymerase such that the error rate of incorrect incorporation of dNTPs is less than one per 1,000 base pairs. Reverse transcription PCR, quantitative reverse transcription PCR, and quantitative real time reverse transcription PCR are other specific examples of PCR. In general, the reaction mixture is subjected to temperature cycles comprising a denaturation stage (typically 80-100° C.), an annealing stage with a temperature that is selected based on the melting temperature (Tm) of the primers and the degeneracy of the primers, and an extension stage (for example 40-75° C.). In real-time PCR analysis, additional reagents, methods, optical detection systems, and devices known in the art are used that allow a measurement of the magnitude of fluorescence in proportion to concentration of amplified template. In such analyses, incorporation of fluorescent dye into the amplified strands may be detected or measured.

Either primers or primers along with probes allow a quantification of the amount of specific template DNA present in the initial sample. In addition, RNA may be detected by PCR analysis by first creating a DNA template from RNA through a reverse transcriptase enzyme (i.e., the creation of cDNA). The marker expression may be detected by quantitative PCR analysis facilitating genotyping analysis of the samples.

The various and non-limiting embodiments of the PCR-based method detecting marker expression level as described herein may comprise one or more probes and/or primers. Generally, the probe or primer contains a sequence complementary to a sequence specific to a region of the nucleic acid of the marker gene. A sequence having less than 60% 70%, 80%, 90%, 95%, 99% or 100% identity to the identified gene sequence may also be used for probe or primer design if it is capable of binding to its complementary sequence of the desired target sequence in marker nucleic acid.

In some embodiments, sample or biological sample may include a bodily tissue, fluid, or any other specimen that may be obtained from a living organism. The term “extraction” as used herein refers to any method for separating or isolating the nucleic acids from a sample, more particularly from a biological sample, such as blood or plasma. Nucleic acids such as RNA or DNA may be released, for example, by cell lysis. Moreover, in some aspects, extraction may also encompass the separation or isolation of extracellular RNAs (e.g., extracellular miRNAs) from one or more extracellular structures, such as exosomes.

Some embodiments of the invention include the extraction of one or more forms of nucleic acids from one or more samples. In some aspects, the extraction of the nucleic acids can be provided using one or more techniques known in the art. In other embodiments, methodologies of the invention can use any other conventional methodology and/or product intended for the isolation of intracellular and/or extracellular nucleic acids (e.g., DNA or RNA).

The term “nucleic acid” or “polynucleotide” as referred to herein comprises all forms of RNA (mRNA, miRNA, rRNA, tRNA, piRNA, ncRNA), DNA (genomic DNA, mtDNA, cfDNA, ctDNA), as well as recombinant RNA and DNA molecules or analogs of DNA or RNA generated using nucleotide analogues. The nucleic acids may be single-stranded or double-stranded. The nucleic acids may include the coding or non-coding strands. The term also comprises fragments of nucleic acids, such as naturally occurring RNA or DNA which may be recovered using one or more extraction methods disclosed herein. “Fragment” refers to a portion of nucleic acid (e.g., RNA or DNA).

As used herein, a “whole genome sequence”, or WGS (also referred to in the art as a “full”, “complete”, or entire” genome sequence), or similar phraseology is to be understood as encompassing a substantial, but not necessarily complete, genome of a subject. In the art the term “whole genome sequence” or WGS is used to refer to a nearly complete genome of the subject, such as at least 95% complete in some usages. The term “whole genome sequence” or WGS as used herein does not encompass “sequences” employed for gene-specific techniques such as single nucleotide polymorphism (SNP) genotyping, for which typically less than 0.1% of the genome is covered. The term “whole genome sequence”, or WGS as used herein does not require that the genome be aligned with any reference sequence, and does not require that variants or other features be annotated. As used herein the term “whole genome sequencing” refers to determining the complete DNA sequence of the genome at one time.

The term “library,” as used herein refers to a library of genome/transcriptome-derived sequences. The library may also have sequences allowing amplification of the “library” by the polymerase chain reaction or other in vitro amplification methods well known to those skilled in the art. In various embodiments, the library may have sequences that are compatible with next-generation high throughput sequencing platforms. In some embodiments, as a part of the sample preparation process, “barcodes” may be associated with each sample. In this process, short oligonucleotides are added to primers, where each different sample uses a different oligo in addition to a primer.

In certain embodiments, primers and barcodes are ligated to each sample as part of the library generation process. Thus during the amplification process associated with generating the ion amplicon library, the primer and the short oligo are also amplified. As the association of the barcode is done as part of the library preparation process, it is possible to use more than one library, and thus more than one sample. Synthetic nucleic acid barcodes may be included as part of the primer, where a different synthetic nucleic acid barcode may be used for each library. In some embodiments, different libraries may be mixed as they are introduced to a flow cell, and the identity of each sample may be determined as part of the sequencing process.

The following examples are given for purely illustrative and non-limiting purposes of the present invention.

### Example

Sample Collection

For this study, a total of 73 dogs, including 28 affected (AF) by CIPF and 45 unaffected (UF) by CIPF, were sampled via saliva samples. The sex ratio (male/females) in affected dogs was 0.86, whereas in unaffected dogs it was 1.26 (p=0.466). The average age for affected dogs was 12.8 years (range: 6.9-17.0), whereas in unaffected dogs it was 12.7 years (range: 9.1-16.8) (p=0.793). Sex information was not available for four samples, and age information was not available for one sample. Saliva was collected by the owner using the Oragene ANIMAL kit (DNAGenotek, Ottawa, CA) and returned to the laboratory at ambient temperature.

DNA Extraction and Whole Genome Sequencing

DNA was isolated from the collected saliva specimens. Construction of the shotgun genomic libraries and sequencing on the NovaSeq 6000 was carried out. DNA was quantitated with the Qubit High Sensitivity reagent (Thermo Fisher, Waltham, Mass.) and diluted with water to 2.5 ng/μL in a total volume of 12 uL. Libraries were prepared with the Riptide DNA library prep kit (iGenomX, Carlsbad, Calif.). Briefly, random primers with 5′ barcoded Illumina adapter sequences (one sequence that is unique to each sample) were annealed to denatured DNA template. A polymerase extended each primer and this action was terminated with a biotinylated dideoxynucleotide, of which there was a small fraction in the nucleotide mix. The biotinylated products were then pooled for all of the samples and captured on streptavidin-coated magnetic beads. A second 5′ adapter-tailed random primer was used with a strand-displacing polymerase to convert the captured DNA strands into a dual adapter library. PCR was used to amplify the products and add an index barcode. These libraries were then sequenced for 150 nt from each side of the DNA fragments (paired-reads) on a NovaSeq 6000 (Illumina, San Diego, Calif., USA) one lane of an S2 flowcell.

Data Analysis

The fastq files were generated with the bcl2fastq v2.20 Conversion Software (Illumina) and demultiplexed with the fgbio tool from Fulcrum Genomics. Sequencing reads were processed and imputed using version 2.0 of the Gencove, Inc. analysis pipeline for canine low-pass sequencing data. Reads were aligned to the reference genome CanFam3.1 using bwa mem v0.7.17 [21] and sorted, and duplicates were marked using samtools v1.8 [22], and imputation performed using loimpute v0.18 (Gencove, Inc) [23]. The imputation reference panel consisted of 676 sequenced dogs across the 91 dog breeds for a total of 53 million sites.

The resulting vcf files of 28 affected and 45 unaffected dogs were filtered using vcftools v0.1.16 [24] including only biallelic, single nucleotide variants (SNVs) and variants with genotype probability (GP, indicating the imputation quality) greater or equal then 0.90. Then, we filtered the dataset with PLINK v1.9 [25] using the following thresholds: SNP genotyping rate ≥95%, minimum allele frequency (MAF)≥5%, Hardy-Weinberg equilibrium in unaffected p≥1.0×10-5, sample genotyping rate ≥90%, and keeping only autosomal variants. Principal component analysis (PCA) was conducted with PLINK v1.9 to detect and remove outliers. Specifically, the identity by similarity (IBS) metric was used taking into account from the first to the fifth closest neighbor, and classifying as outliers samples with Z≤−4, representing 4 standard deviations below the group mean. After outlier removal, the original dataset including only high-quality imputed SNPs was filtered again with PLINK v1.9 using the same thresholds. Identity by descent (IBD) analysis was conducted to estimate the relatedness between all the pair of samples calculating the pi-hat value using the—genome command in PLINK v1.9. This analysis was conducted as additional quality control (i.e., identification of duplicated samples), and the adjustment for relatedness in the GWAS was conducted using the relatedness matrix computed with GEMMA v0.96 [26,27].

The GWAS was conducted using a mixed linear model (MLM) to account for relatedness and population structure, as implemented in the GEMMA v0.96 software, assessing the significance with the Wald test. The first step of the analysis included the estimation of the relatedness matrix, and the top 10 principal components. These metrics were included in the second step (GWAS), allowing for the adjustment for both relatedness and population structure. Results were corrected at the genome-wide level using the Bonferroni method, accounting for the number of independent SNPs tested according the linkage disequilibrium (LD) patterns estimated using the option—indep-pairwise 10,000 1 0.80 in PLINK v1.9. Using this approach, 101,740 independent SNPs were found. Variants were annotated according CanFam3.1 assembly using the R-package Biomart v2.42.0 [28]. Lambda inflation factor and quantile-quantile plots (qqplots) were computed using the R-package snpStats.

The data was further analyzed by conducting a gene-based association analysis using the GATES method [29], using as input the summary statistics obtained from the GEMMA analysis. First, we filtered the dataset including the SNPs located at +/−1500 bp from each gene to include the variants located in the promoter and in the 3′ regions. The analysis was also conducted including larger regions (+/−5000 and +/−10,000 bp). Ensemble start and end coordinates of each gene were retrieved using the R-package Biomart v2.42.0, according the dog assembly CanFam3.1. The Ensembl Biomart gene coordinates correspond to the outermost transcript start and end. Then, for each gene, we computed a matrix of correlation between SNPs using the unaffected samples in order to account for the linkage disequilibrium. The correlation matrix was computed with the Pearson's method using the “cor” function implemented in R, using the option use=“na.or.complete” to deal with missing genotypes. Finally, the GATES statistics were computed using as input the p-values from the MLM analysis and the correlation linkage disequilibrium matrix. The analysis was conducted using the GATES2 function as implemented in the R-package aSPU. P-values were corrected using the Bonferroni method adjusting for the total number of genes tested.

Results from the both SNP and gene level analysis were compared with a list of 41 genes compiled from the largest and most recent human IPF GWAS [30,31]. Allele frequencies were compared using a general dog population reference dataset including several breeds [32].

Results

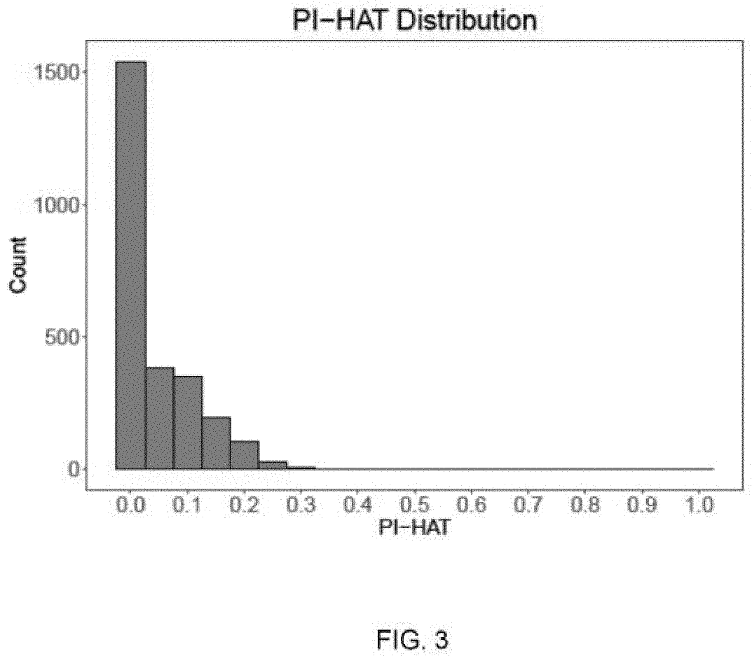

After imputation, INDELS, low quality variants, and variants with more than two alleles were removed. A median number of SNVs per sample were obtained equal to 35,916,311 (range: 33,918,432-36,243,510). Samples showed a median depth of 1×. The median value of variants covered with equal to or greater than five reads per sample was 1,902,261 (range: 34,490-12,200,995). The dataset was filtered with PLINK v1.9, obtaining 1,839,683 variants for study with an average genotyping rate equal to 98.0%. PCA and IBS analyses was conducted to identify significant outliers and identified one outlier, on the basis of the first and second principal components, with a Z score <−4. This outlier sample was removed from the analysis and the dataset was filtered again, obtaining 1,843,695 SNPs in 28 affected and 44 unaffected animals. The PCA and IBS analyses were then repeated and further outliers were not found. Affected and unaffected sample groups were not statistically different for sex (p=0.466) or age (p=0.793). The IBD analysis demonstrated a pi-hat=0.043+/−0.068 (range: 0.000-0.500). The distribution of pi-hat values for each sample pair is shown in FIG. 3.

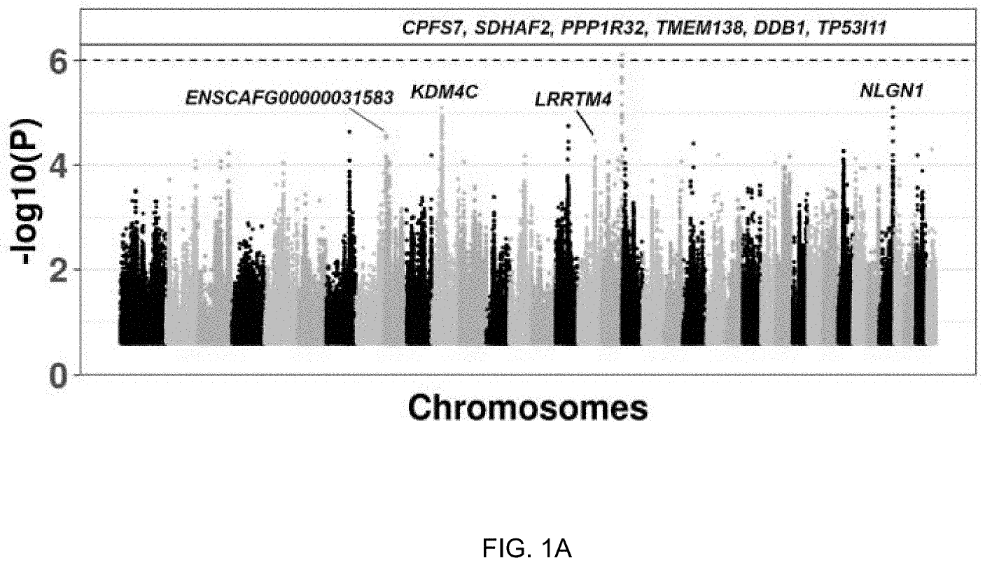

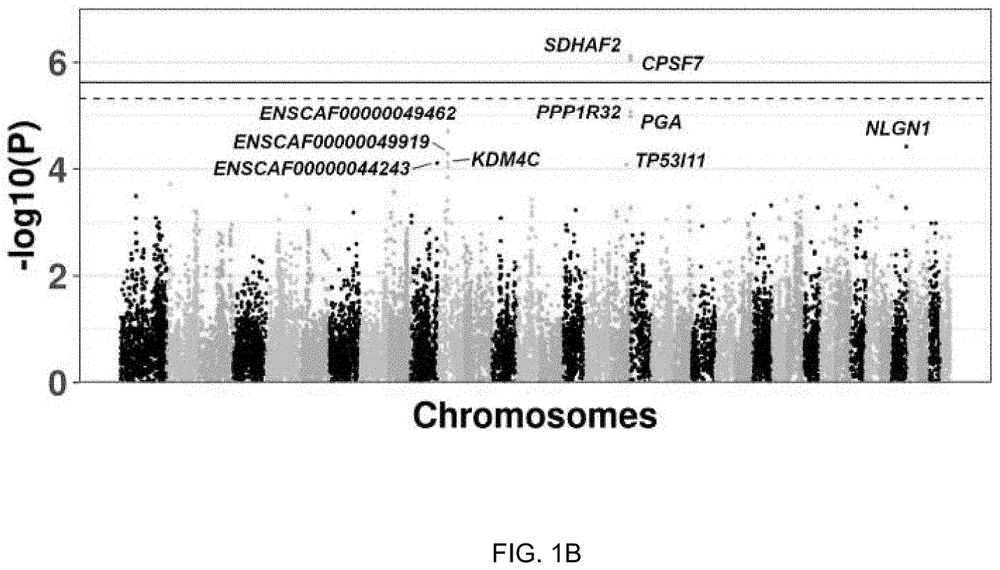

We ran the GWAS accounting for relatedness and population stratification using the linear mixed model as implemented in the GEMMA software [26,27]. Age and sex were not included as covariates in the model because these factors did not differ significantly between the affected and unaffected animals. The results were adjusted (adj) with the Bonferroni method accounting for the 101,740 independent SNPs estimated by regional linkage disequilibrium patterns, setting the genome-wide significance threshold at p<4.91×10-7 (adj p<0.05). A level of p<9.83×10-7 (adj p<0.10) were considered as “suggestive” association. The top 10 variants ranked by adj p-value are reported in Table 1, and the Manhattan plot with the top 500,000 SNPs is illustrated in FIG. 1A.

FIG. 1A shows a plot of the top 500,000 SNPs ranked by unadjusted p-value. In FIG. 1A, the continuous and dashed lines indicate the genome-wide (p<4.91×10-7) and suggestive (p<9.83×10-7) significance thresholds, respectively. The p-value adjustment was conducted using the Bonferroni method, accounting for 101,740 independent SNPs estimated using regional linkage disequilibrium (LD) patterns. Gene names shown in FIG. 1A are the top 10 according to the SNP level analysis.

TABLE 1 shows details of the top 10 single nucleotide polymorphisms (SNPs) detected in the genome-wide association study (GWAS). In TABLE 1, “A1” is the minor frequency allele referred to the total sample, “A2” is the major frequency allele referred to the total sample; “FA” is the frequency of A1 in affected samples; “FU” is frequency of A2 in unaffected samples; “u” indicates the variant is located upstream, “i” indicates the variant is located in an intron; “5′ UTR” indicates the variant is located in the 5′ untranslated region (UTR).

TABLE 1

| | | | | | | | | | | | Gene | Consequence |
|---|---|---|---|---|---|---|---|---|---|---|---|---|
| Refsnp ID | BP | A1 | A2 | FA | FU | Depth (SD) | Beta | p | Adj p | Ensembl Gene ID | Name | Type |
| rs22669389 | 54992254 | T | A | 0.704 | 0.333 | 0.451 ± 0.713 | 0.406 | 7.7 × 10⁻⁷ | 0.078 | ENSCAFG00000030303; | SDHAF2; | u, i; u, i |
| | | | | | | | | | | ENSCAFG00000016152 | CPSF7 | |
| rs22647286 | 54987884 | C | T | 0.704 | 0.333 | 3.183 ± 2.875 | 0.394 | 1.2 × 10⁻⁶ | 0.124 | ENSCAFG00000030303; | SDHAF2; | u, i; u, i |
| | | | | | | | | | | ENSCAFG00000016152 | CPSF7 | |
| rs851654341 | 54986491 | A | G | 0.704 | 0.333 | 2.324 ± 2.123 | 0.394 | 1.2 × 10⁻⁶ | 0.124 | ENSCAFG00000030303; | SDHAF2; | u, i; u, i |
| | | | | | | | | | | ENSCAFG00000016152 | CPSF7 | |
| rs852097932 | 54986070 | A | G | 0.704 | 0.337 | 2.861 ± 2.209 | 0.394 | 1.3 × 10⁻⁶ | 0.131 | ENSCAFG00000030303; | SDHAF2; | u, i; u, i |
| | | | | | | | | | | ENSCAFG00000016152 | CPSF7 | |
| rs22686152 | 54992285 | A | G | 0.704 | 0.345 | 0.732 ± 0.940 | 0.386 | 2.1 × 10⁻⁶ | 0.213 | ENSCAFG00000030303; | SDHAF2; | u, i; u, i |
| | | | | | | | | | | ENSCAFG00000016152 | CPSF7 | |
| rs22647289 | 54987464 | G | T | 0.704 | 0.345 | 5.423 ± 3.702 | 0.391 | 2.1 × 10⁻⁶ | 0.214 | ENSCAFG00000030303; | SDHAF2; | 5′ UTR, u; |
| | | | | | | | | | | ENSCAFG00000016152 | CPSF7 | 5′ UTR, u |
| rs850942449 | 54983627 | A | G | 0.704 | 0.345 | 2.831 ± 2.449 | 0.393 | 2.2 × 10⁻⁶ | 0.223 | ENSCAFG00000030303; | SDHAF2; | u, i; u, i |
| | | | | | | | | | | ENSCAFG00000016152 | CPSF7 | |
| — | 54984004 | G | A | 0.704 | 0.345 | 0.887 ± 0.919 | 0.393 | 2.2 × 10⁻⁶ | 0.223 | ENSCAFG00000030303 | SDHAF2 | i |
| rs22647283 | 54987912 | C | T | 0.692 | 0.326 | 2.535 ± 2.709 | 0.390 | 2.6 × 10⁻⁶ | 0.263 | ENSCAFG00000030303; | SDHAF2; | u, i; u, i |
| | | | | | | | | | | ENSCAFG00000016152 | CPSF7 | |
| rs850871193 | 54986170 | C | T | 0.692 | 0.337 | 3.028 ± 2.646 | 0.387 | 4.1 × 10⁻⁶ | 0.413 | ENSCAFG00000030303; | SDHAF2; | u, i; u, i |
| | | | | | | | | | | ENSCAFG00000016152 | CPSF7 | |

The Lambda inflation factor obtained was 1.052, demonstrating an absence of significant population stratification after principal components adjustment. One variant, rs22669389, corresponding to position 54992254 on canine (CanFam3.1) chromosome 18, was identified at a suggestive level of significance (adj p=0.078). Thus, the presence of a T allele at position 54992254 on canine chromosome 18 was found to be associated with CIPF.

All of the top SNPs reported in TABLE 1 are located in the same region on chromosome 18 between 54,983,627 and 54,992,285 (8658 bp), encompassing the two overlapping genes “succinate dehydrogenase complex assembly factor 2” (SDHAF2) and “cleavage and polyadenylation specific factor 7” (CPSF7), and being located upstream, in introns or in 5′ untranslated regions (UTR) of the two genes. In addition to the SNP-level analysis, a multi-marker test was computed using the GATES method, adjusting the results for the total number of genes tested (n=18,110; p<2.76×10\hat{ }−6). SNPs were assigned to a gene when the SNP was found within 1500 bp from the gene. The results showed two significant genes after Bonferroni correction: CPSF7 (adj p=0.016) and SDHAF2 (adj p=0.024). The corresponding Manhattan plot is shown in FIG. 1B.

FIG. 1B shows a Manhattan plot of the genes ranked by p-value. In FIG. 1B, the continuous and dashed lines indicate the genome-wide and suggestive significance thresholds, respectively. The adjustment was conducted using the Bonferroni method accounting for the total number of genes tested (n=18,110). Gene names shown in FIG. 1B are the top 10 according the gene level analysis.

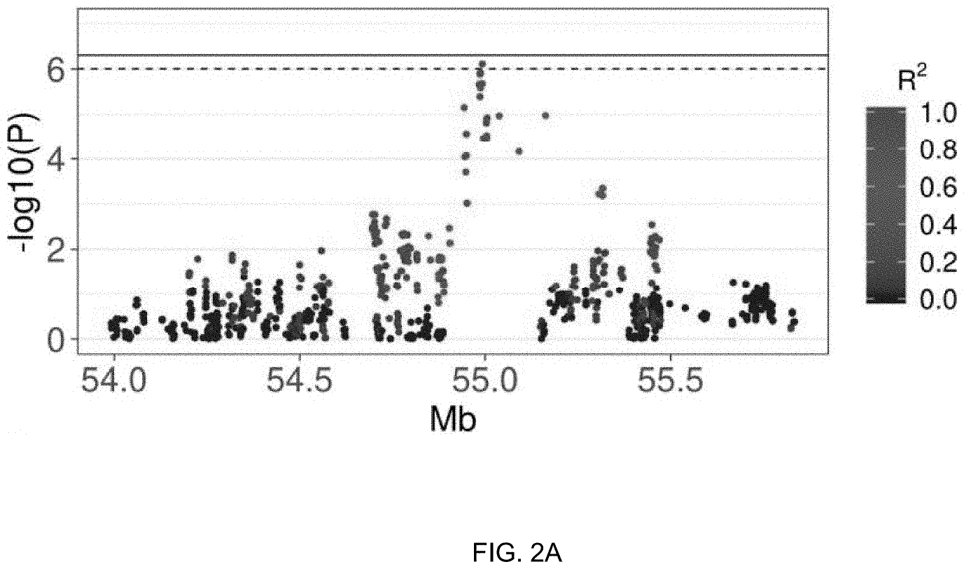

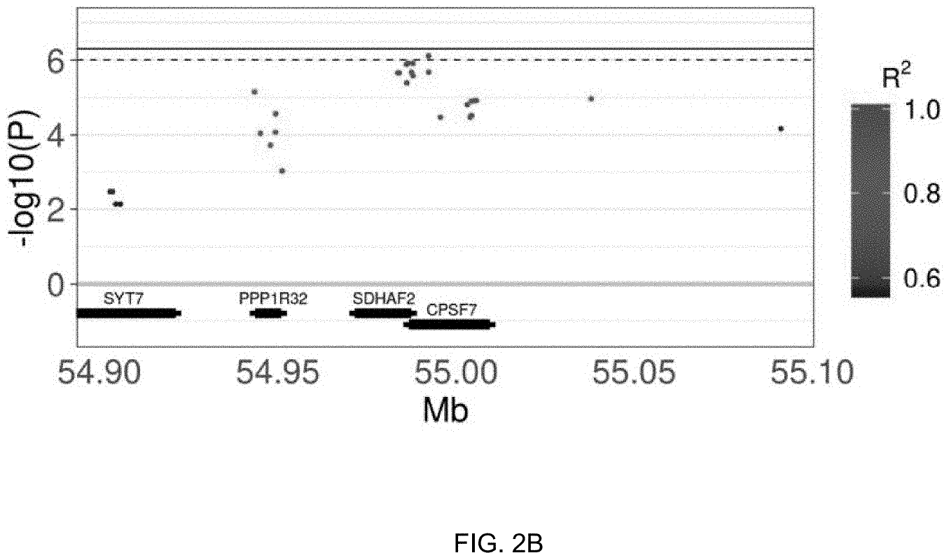

The results of the GATES analysis were confirmed when larger distances were considered for the SNP to gene assignments (for both +/−5000 bp and +/−10,000 bp). The regional plot including all of the SNPs in the region is shown in FIGS. 2A and 2B, also reporting the linkage disequilibrium patterns as R2 values.

In FIGS. 2A and 2B, the continuous lines indicate the genome-wide significance threshold and the dashed lines indicate the suggestive significance thresholds. The shade of the points indicates the linkage disequilibrium (expressed as R2) between the top (rs22669389) and the close SNPs. Values of R2 range from 0 (absence of LD) to 1 (complete LD). FIG. 2A shows a region of 2 Mb around the top SNP (rs22669389), with the R2 ranging from 0 (absence of LD) to 1 (complete LD). FIG. 2B shows a smaller region around the top SNP (0.2 Mb), due to the closeness of the top SNP, showing the R2 ranging from 0.56 to 1. Thick sections of the genes represent the actual gene region according to Ensemble, the thin sections represent the surrounding regions (+/−1500).

Considering the two largest and most recent human IPF GWAS, a list of 41 candidate genes was compiled and compared to the results [30,31]. The list included signals detected in the two studies, as well as genes identified in previous studies tested for validation purposes. These results included all SNPs or genes (GATES analysis) with p<0.05, (n=104,370 SNPs), and a number of genes ranging from 2614 to 3616, depending on the gene flanking region used (+/−1500 bp, +/−5000 bp, and +/−10,000 bp). We detected nine genes showing SNPs with p<0.05, and nine genes showing at least one significant result in the GATES analysis, five of them overlapping with the SNP analysis (CD1C, DEPTOR, MAD1L1, MRPL13, and MUC5B). All the genes in our study showed p<0.05, with the exception of MUC5B, which showed p<0.01.

Allele frequencies of the top SNPs in TABLE 1 were compared with a reference dataset generated for imputation purposes, including a whole genome data from 365 dogs from different breeds [32]. The allele frequencies of non-WHWT (n=362) were observed as similar to the affected dogs in our study, with the exception of one SNP. Additionally, allele frequencies of the WHWT (n=3) were in the range of average allele frequency in these results.

A significant genetic risk factors for CIPF in the West Highland White Terrier dog breed was detected using a GWAS including 1,839,683 informative SNPs. Applying a gene-level approach, genome-wide significant signals were observed in CPSF7 (cleavage and polyadenylation specific factor 7) and SDHAF2 (succinate dehydrogenase complex assembly factor 2) (adj p=0.016 and adj p=0.024, respectively). These two overlapping genes include 15 and 8 SNPs, respectively. Each of the top associated SNPs were located in introns, 5′UTR, or upstream of the two genes.

CPSF7 is a human orthologue (gene order conservation score=100), and encodes for the 59 kDa subunit of Cleavage Factor Im, involved in the cleavage and polyadenylation of pre-mRNAs. The CPSF7 gene is related to several mRNA process pathways, such as “mRNA splicing”, “metabolism of RNA”, “mRNA 3′-end processing”, “processing of capped intronless pre-mRNA”, and “RNA polymerase II transcription”. CPSF7 was found to be involved in lung adenocarcinoma (LAD). Specifically, Spl Transcription Factor (SP1) induces the promoter activity of LINC00958, which, when overexpressed, drives LAD progression via the miR-625-5p/CPSF7 axis [33]. The genetic association discussed herein might reveal the importance of CPSF7 in CIPF, through the same pathologic mechanism as in lung cancer. IPF in humans is a risk factor for lung cancer, increasing the chance of development from 7% to 10% [34]. Additionally, there are several genetic, molecular, and cellular mechanisms shared between lung fibrosis and lung cancer such as myofibroblast activation, endoplasmatic reticulum stress, alteration of growth factor expression, and genetic and epigenetic variations [34]. CPFS7 has also associated with liver cancer [35].

SDHAF2 encodes a mitochondrial protein involved in the flavination of a succinate dehydrogenase complex subunit and it has largely been associated with paragangliomas in previous literature [36,37].

These results were compared with the two largest and most recent human IPF GWAS [30,31], finding in our study a total of 13 genes with p<0.05 that overlapped with the human candidate gene list. Five genes were detected at both the SNP and gene level analyses (CD1C, DEPTOR, MAD1L1, MRPL13, and MUC5B). However, the genes showed a weak significance (p<0.05), with the exception of mucin 5B, oligomeric mucus/gel-forming (MUC5B) which showed a significance level of p<0.01.

In conclusion, we report for the first time the identification of genetic variants associated with CIPF in the West Highland White Terrier dog breed, located in a region encompassing the CPFS7 and SDHAF2 genes. Our findings demonstrated some overlap with biological functions, with compelling links to previously demonstrated findings in lung cancer, sharing several biological and genetic features with IPF in humans.

As disclosed in the present invention, the minor allele variant SNPs in TABLE 1, or combinations of the minor allele variants SNPs in TABLE 1, can be used for IPF identification, diagnosis, and treatment in a human or canine subject. Presence of one or more of the minor alleles (A1; risk alleles) in TABLE 1 may indicate a subject is at risk of developing IPF or the subject currently has IPF (or CIPF, in the case of canines).

By determining a subject's relative risk for IPF or a diagnosis of IPF, the treatment may include prescribing a therapeutic regimen to treat, prevent or delay onset of IPF. Additionally, knowledge of the risk of developing CIPF may inform canine breeders about whether to consider a canine subject as a potential candidate for breeding.

Treatments for IPF or CIPF may comprise oxygen therapy, pulmonary rehabilitation including exercise training or breathing exercises, organ transplantation, drug therapy, and/or autoantibody reductive therapy. A treatment comprising drug therapy may include administering an effective amount of a pharmaceutical composition, which may include an anti-fibrotic drug or an anti-inflammatory drug. A treatment may comprise administering an effective amount of a brohchodilator or a steroid. A treatment may comprise administering an effective amount of an immunosuppressant.

A treatment may comprise administering an effective amount of a pharmaceutical composition, which may include Nintedanib, Pirfenidone, prednisone, Mycophenolate mofetil, mycophenolic acid, Azathioprine, pamrevlumab, omeprazole, cyclophosphamide, TRK-250 (suppression of the expression of transforming growth factor beta 1 protein), FG-3019, ART-123, TD139 (a galectin 3 inhibitor), KD025, GKT137831 (an inhibitor of nicotinamide adenine dinucleotide phosphate (NADPH) oxidase (NOX) isoforms), GSK3008348, BG00011, GLPG1205, GLPG1690, low dose carbon monoxide (CO), and/or VAY736.

The concept of a pharmaceutical composition includes one or more of the disclosed compounds or a pharmaceutically acceptable salt thereof with or without any other additive/pharmaceutically acceptable excipient. The physical form of the invention may affect the route of administration and one skilled in the art would know to choose a route of administration that takes into consideration both the physical form of the compound and the disorder to be treated. Pharmaceutical compositions that include the disclosed compounds may be prepared using methodology well known in the pharmaceutical art.

Pharmaceutical compositions, including the one or more disclosed compounds, may include materials capable of modifying the physical form of a dosage unit (e.g., pharmaceutically acceptable excipients). In one non-limiting example, the composition includes a material that forms a coating that contains the one or more disclosed compounds. Materials that may be used in a coating include, for example, sugar, shellac, gelatin, or any other inert coating agent.

Pharmaceutical compositions including the one or more disclosed compounds may be prepared as a gas or aerosol. Aerosols encompass a variety of systems including colloids and pressurized packages. Delivery of a composition in this form may include propulsion of a pharmaceutical composition including the one or more disclosed compounds through use of liquefied gas or other compressed gas or by a suitable pump system. Aerosols may be delivered in single phase, bi-phasic, or multi-phasic systems.

In some aspects of the invention, the pharmaceutical composition including the one or more disclosed compounds is in the form of a solvate. Such solvates are produced by the dissolution of the one or more disclosed compounds in a pharmaceutically acceptable solvent. Pharmaceutically acceptable solvents include any mixtures of one or more solvents. Such solvents may include pyridine, chloroform, propan-1-ol, ethyl oleate, ethyl lactate, ethylene oxide, water, ethanol, and any other solvent that delivers a sufficient quantity of the one or more disclosed compounds to treat the indicated condition.

Treatment of a condition, such as treatment of a subject having one or more of the disclosed markers for IPF or CIPF, is the practice of any method, process, or procedure with the intent of halting, inhibiting, slowing or reversing the progression of a disease, disorder, or condition, substantially ameliorating clinical symptoms of a disease, disorder, or condition substantially preventing the appearance of clinical symptoms of a disease, disorder or condition, up to and including returning the diseased entity to its condition prior to the development of the disease.

Pharmaceutical compositions that include the one or more disclosed compounds may also include at least one pharmaceutically acceptable carrier/excipient. As used herein, “carrier(s)” can be used interchangeably with “excipient(s)” Carriers include any substance that may be administered with the one or more disclosed compounds with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, and oils (including petroleum, animal, vegetable or synthetic oils.) Such carriers include particulates such as a tablet or powder, liquids such as oral syrup or injectable liquid, and inhalable aerosols. Further examples include saline, gum acacia, gelatin, starch paste, talc, keratin, colloidal silica, and urea. Such carriers may further include binders such as ethyl cellulose, carboxymethylcellulose, microcrystalline cellulose, or gelatin; excipients such as starch, lactose or dextrins; disintegrating agents such as alginic acid, sodium alginate. Primogel, and corn starch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of carriers include polyethylene glycol, cyclodextrin, oils, or any other similar liquid carrier that may be formulated into a capsule. Still further examples of carriers include sterile diluents such as water for injection, saline solution, physiological saline. Ringer's solution, isotonic sodium chloride, fixed oils such as synthetic mono or diglycerides, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents.

The pharmaceutical composition, including the one or more disclosed compounds, may take any of a number of formulations depending on the physicochemical form of the composition and the type of administration. Such forms include solutions, suspensions, emulsions, tablets, pills, pellets, capsules, capsules including liquids, powders, sustained-release formulations, directed release formulations, lyophylates, suppositories, emulsions, aerosols, sprays, granules, powders, syrups, elixirs, or any other formulation now known or yet to be disclosed. Additional examples of suitable pharmaceutical carriers and formulations are well known in the art.

Methods of administration include, but are not limited to, oral administration and parenteral administration. Parenteral administration includes, but is not limited to intradermal, intramuscular, intraperitoneal, intravenous, subcutaneous, intrathecal, intranasal, epidural, sublingual, intramsal, intracerebral, iratraventricular, intrathecal, intravaginal, transdermal, rectal, by inhalation, or topically to the ears, nose, eyes, or skin. Other methods of administration include but are not limited to infusion techniques including infusion or bolus injection, by absorption through epithelial or mucocutaneous linings such as oral mucosa, rectal and intestinal mucosa. Compositions for parenteral administration may be enclosed in ampoule, a disposable syringe or a multiple-dose vial made of glass, plastic or other material.

Administration may be systemic or local. Local administration is administration of the disclosed compound to the area in need of treatment (e.g., areas of the respiratory tract, including the nasal cavity, the trachea, the lungs, the bronchi, etc.). Examples include local infusion during surgery; topical application, by local injection; by a catheter; by a suppository; or by an implant. Administration may be by direct injection into the central nervous system by any suitable route, including intraventricular and intrathecal injection. Intraventricular injection can be facilitated by an intraventricular catheter, for example, attached to a reservoir, such as an Ommaya reservoir. Pulmonary administration may be achieved by any of a number of methods known in the art. Examples include the use of an inhaler or nebulizer, formulation with an aerosolizing agent, or via perfusion in a fluorocarbon or synthetic pulmonary surfactant. The disclosed compound may be delivered in the context of a vesicle such as a liposome or any other natural or synthetic vesicle. Additional examples of suitable modes of administration are well known in the art.

A pharmaceutical composition formulated to be administered by injection may be prepared by dissolving the one or more disclosed compounds with water so as to form a solution. In addition, a surfactant may be added to facilitate the formation of a homogeneous solution or suspension. Surfactants include any complex capable of non-covalent interaction with the disclosed compound so as to facilitate dissolution or homogeneous suspension of the compound.

Pharmaceutical compositions including the one or more disclosed compounds may be prepared in a form that facilitates topical or transdermal administration. Such preparations may be in the form of a solution, emulsion, ointment, gel base, transdermal patch or iontophoresis device. Examples of bases used in such compositions include petrolatum, lanolin, polyethylene glycols, beeswax, mineral oil, diluents such as water and alcohol, and emulsifiers and stabilizers, thickening agents, or any other suitable base now known or yet to be disclosed.

Determination of an effective amount of the one or more disclosed compounds is within the capability of those skilled in the art, especially in light of the detailed disclosure provided herein. The effective amount of a pharmaceutical composition used to affect a particular purpose as well as its toxicity, excretion, and overall tolerance may be determined in vitro, or in vivo, by pharmaceutical and toxicological procedures either known now by those skilled in the art or by any similar method yet to be disclosed. One example is the in vitro determination of the IC50 (half maximal inhibitory concentration) of the pharmaceutical composition in cell lines or target molecules. Another example is the in vivo determination of the LD50 (lethal dose causing death in 50% of the tested animals) of the pharmaceutical composition. The exact techniques used in determining an effective amount will depend on factors such as the type and physical/chemical properties of the pharmaceutical composition, the property being tested, and whether the test is to be performed in vitro or in vivo. The determination of an effective amount of a pharmaceutical composition will be well known to one of skill in the art who will use data obtained from any tests in making that determination. Determination of an effective amount of disclosed compound for administration also includes the determination of an effective therapeutic amount and a pharmaceutically acceptable dose, including the formulation of an effective dose range for use in vivo, including in humans.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

The publications discussed herein are provided solely for their disclosure prior to the filing date of the present application. Nothing herein is to be construed as an admission that the present invention is not entitled to antedate such publication by virtue of prior invention.
1. Heikkilä, H. P.; Lappalainen, A. K.; Day, M. J.; Clercx, C.; Rajamäki, M. M. Clinical, bronchoscopic, histopathologic, diagnostic imaging, and arterial oxygenation findings in west highland white terriers with idiopathic pulmonary fibrosis. J. Vet. Intern. Med. 2011, 25, 433-439.
2. Clercx, C.; Fastrés, A.; Roels, E. Idiopathic pulmonary fibrosis in West Highland white terriers: An update. Vet. J. 2018, 242, 53-58.
3. Heikkilä-Laurila, H. P.; Rajamäki, M. M. Idiopathic pulmonary fibrosis in west highland white terriers. Vet. Clin. N. Am. Small Anim. Pract. 2014, 44, 129-142.
4. Daccord, C.; Maher, T. M. Recent advances in understanding idiopathic pulmonary fibrosis. F1000Research 2016, 5, 1046.
5. Coward, W. R.; Saini, G.; Jenkins, G. The pathogenesis of idiopathic pulmonary fibrosis. Ther. Adv. Respir. Dis. 2010, 4, 367-388.
6. Lilja-Maula, L.; Syriä, P.; Laurila, H. P.; Sutinen, E.; Palviainen, M.; Ritvos, O.; Koli, K.; Rajamäki, M. M.; Myllärniemi, M. Upregulation of alveolar levels of activin B, but not activin A, in lungs of west highland white terriers with idiopathic pulmonary fibrosis and diffuse alveolar damage. J. Comp. Pathol. 2015, 152, 192-200.
7. Krafft, E.; Lybaert, P.; Roels, E.; Laurila, H. P.; Rajamäki, M. M.; Farnir, F.; Myllärniemi, M.; Day, M. J.; Mc Entee, K.; Clercx, C. Transforming Growth Factor Beta 1 Activation, Storage, and Signaling Pathways in Idiopathic Pulmonary Fibrosis in Dogs. J. Vet. Intern. Med. 2014, 28, 1666-1675.
8. Krafft, E.; Laurila, H. P.; Peters, I. R.; Bureau, F.; Peeters, D.; Day, M. J.; Rajamäki, M. M.; Clercx, C. Analysis of gene expression in canine idiopathic pulmonary fibrosis. Vet. J. 2013, 198, 479-486.
9. Krafft, E.; Heikkilä, H. P.; Jespers, P.; Peeters, D.; Day, M. J.; Rajamäki, M. M.; Mc Entee, K.; Clercx, C. Serum and Bronchoalveolar Lavage Fluid Endothelin-1 Concentrations as Diagnostic Biomarkers of Canine Idiopathic Pulmonary Fibrosis. J. Vet. Intern. Med. 2011, 25, 990-996.
10. Noth, I.; Zhang, Y.; Ma, S. F.; Flores, C.; Barber, M.; Huang, Y.; Broderick, S. M.; Wade, M. S.; Hysi, P.; Scuirba, J.; et al. Genetic variants associated with idiopathic pulmonary fibrosis susceptibility and mortality: A genome-wide association study. Lancet Respir. Med. 2013, 1, 309-317.
11. Fingerlin, T. E.; Murphy, E.; Zhang, W.; Peljto, A. L.; Brown, K. K.; Steele, M. P.; Loyd, J. E.; Cosgrove, G. P.; Lynch, D.; Groshong, S.; et al. Genome-wide association study identifies multiple susceptibility loci for pulmonary fibrosis. Nat. Genet. 2013, 45, 613-620.
12. Mushiroda, T.; Wattanapokayakit, S.; Takahashi, A.; Nukiwa, T.; Kudoh, S.; Ogura, T.; Taniguchi, H.; Kubo, M.; Kamatani, N.; Nakamura, Y. A genome-wide association study identifies an association of a common variant in TERT with susceptibility to idiopathic pulmonary fibrosis. J. Med. Genet. 2008, 45, 654-656.
13. Shearin, A. L.; Ostrander, E. A. Leading the way: Canine models of genomics and disease. Dis. Model. Mech. 2010, 3, 27-34.
14. Wayne, R. K.; Ostrander, E. A. Lessons learned from the dog genome. Trends Genet. 2007, 23, 557-567.
15. Lindblad-Toh, K.; Wade, C. M.; Mikkelsen, T. S.; Karlsson, E. K.; Jaffe, D. B.; Kamal, M.; Clamp, M.; Chang, J. L.; Kulbokas, E. J.; Zody, M. C.; et al. Genome sequence, comparative analysis and haplotype structure of the domestic dog. Nature 2005, 438, 803-819.
16. Ostrander, E. A.; Kruglyak, L. Unleashing the canine genome. Genome Res. 2000, 10, 1271-1274.
17. Gallana, M.; Utsunomiya, Y. T.; Dolf, G.; Pintor Torrecilha, R. B.; Falbo, A. K.; Jagannathan, V.; Leeb, T.; Reichler, I.; Sölkner, J.; Schelling, C. Genome-wide association study and heritability estimate for ectopic ureters in Entlebucher mountain dogs. Anim. Genet. 2018, 49, 645-650.
18. Peiravan, A.; Bertolini, F.; Rothschild, M. F.; Simpson, K. W.; Jergens, A. E.; Allenspach, K.; Werling, D. Genome-wide association studies of inflammatory bowel disease in German shepherd dogs. PLoS ONE 2018, 13, e0200685.
19. Gast, A. C.; Metzger, J.; Tipold, A.; Distl, O. Genome-wide association study for hereditary ataxia in the Parson Russell Terrier and DNA-testing for ataxia-associated mutations in the Parson and Jack Russell Terrier. BMC Vet. Res. 2016, 12, 225.
20. Bianchi, M.; Dahlgren, S.; Massey, J.; Dietschi, E.; Kierczak, M.; Lund-Ziener, M.; Sundberg, K.; Thoresen, S. I.; Kampe, O.; Andersson, G.; et al. A multi-breed genome-wide association analysis for canine Hypothyroidism identifies a shared major risk locus on CFA12. PLoS ONE 2015, 10, e0134720.
21. Li, H.; Durbin, R. Fast and accurate short read alignment with Burrows-Wheeler transform. Bioinformatics 2009, 25, 1754-1760.
22. Li, H.; Handsaker, B.; Wysoker, A.; Fennell, T.; Ruan, J.; Homer, N.; Marth, G.; Abecasis, G.; Durbin, R. The Sequence Alignment/Map format and SAMtools. Bioinformatics 2009, 25, 2078-2079.
23. Li, N.; Stephens, M. Modelling linkage disequilibrium and identifying recombination hotspots using SNP data genetics. Genetics 2003, 165, 2213-2233.
24. Danecek, P.; Auton, A.; Abecasis, G.; Albers, C. A.; Banks, E.; DePristo, M. A.; Handsaker, R. E.; Lunter, G.; Marth, G. T.; Sherry, S. T.; et al. The variant call format and VCFtools. Bioinformatics 2011, 27, 2156-2158.
25. Purcell, S.; Neale, B.; Todd-Brown, K.; Thomas, L.; Ferreira, M. A.; Bender, D.; Maller, J.; Sklar, P.; de Bakker, P. I. E.; Daly, M. J.; et al. PLINK: a tool set for whole-genome association and population-based linkage analyses. Am. J. Hum. Genet. 2007, 81, 559-575.
26. Zhou, X.; Stephens, M. Genome-wide efficient mixed-model analysis for association studies. Nat. Genet. 2012, 44, 821-824.
27. Zhou, X.; Stephens, M. Efficient multivariate linear mixed model algorithms for genome-wide association studies. Nat. Methods 2014, 11, 407-409.
28. Durinck, S.; Spellman, P. T.; Birney, E.; Huber, W. Mapping identifiers for the integration of genomic datasets with the R/Bioconductor package biomaRt. Nat. Protoc. 2009, 4, 1184-1191.
29. Li, M.-X.; Gui, H.-S.; Kwan, J. S. H.; Sham, P. C. GATES: A rapid and powerful gene-based association test using extended Simes procedure. Am. J. Hum. Genet. 2011, 88, 283-293.
30. Allen, R. J.; Porte, J.; Braybrooke, R.; Flores, C.; Fingerlin, T. E.; Oldham, J. M.; Guillen-Guio, B.; Ma, S. F.; Okamoto, T.; John, A. E.; et al. Genetic variants associated with susceptibility to idiopathic pulmonary fibrosis in people of European ancestry: A genome-wide association study. Lancet Respir. Med. 2017, 5, 869-880.
31. Allen, R. J.; Guillen-Guio, B.; Oldham, J. M.; Ma, S. F.; Dressen, A.; Paynton, M. L.; Kraven, L. M.; Obeidat, M.; Li, X.; Ng, M.; et al. Genome-wide association study of susceptibility to idiopathic pulmonary fibrosis. Am. J Respir. Crit. Care Med. 2020, 201, 564-574.
32. Hayward, J. J.; White, M. E.; Boyle, M.; Shannon, L. M.; Casal, M. L.; Castelhano, M. G.; Center, S. A.; Meyers-Wallen, V. N.; Simpson, K. W.; Sutter, N. B.; et al. Imputation of canine genotype array data using 365 whole-genome sequences improves power of genome-wide association studies. PLOS Genet. 2019, 15, e1008003.
33. Yang, L.; Li, L.; Zhou, Z.; Liu, Y.; Sun, J.; Zhang, X.; Pan, H.; Liu, S. SP1 induced long non-coding RNA LINC00958 overexpression facilitate cell proliferation, migration and invasion in lung adenocarcinoma via mediating miR-625-5p/CPSF7 axis. Cancer Cell Int 2020, 20, 24.
34. Ballester, B.; Milara, J.; Cortijo, J. Idiopathic Pulmonary Fibrosis and Lung Cancer: Mechanisms and Molecular Targets. Int. J. Mol. Sci. 2019, 20, 593.
35. Fang, S.; Zhang, D.; Weng, W.; Lv, X.; Zheng, L.; Chen, M.; Fan, X.; Mao, J.; Mao, C.; Ye, Y.; et al. CPSF7 regulates liver cancer growth and metastasis by facilitating WWP2-FL and targeting the WWP2/PTEN/AKT signaling pathway. Biochim. Biophys. Acta Mol. Cell Res. 2020, 1867, 118624.
36. Bausch, B.; Schiavi, F.; Ni, Y.; Welander, J.; Patocs, A.; Ngeow, J.; Wellner, U.; Malinoc, A.; Taschin, E.; Barbon, G.; et al. Clinical characterization of the pheochromocytoma and paraganglioma susceptibility genes SDHA, TMEM127, MAX, and SDHAF2 for gene-informed prevention. JAMA Oncol. 2017, 3, 1204-1212.
37. Smith, J. D.; Harvey, R. N.; Dan, O. A.; Prince, M. E.; Bradford, C. R.; Wolf, G. T.; Else, T.; Basura, G. J. Head and neck paragangliomas: A two-decade institutional experience and algorithm for management. Laryngoscope Investig. Otolaryngol. 2017, 2, 380-389.
38. Corcoran, B. M.; Cobb, M.; Martin, M. W. S.; Dukes-McEwan, J.; French, A.; Luis Fuentes, V.; Boswood, A.; Rhind, S. Chronic pulmonary disease in West Highland white terriers. Vet. Rec. 1999, 144, 611-616.
39. Roels, E.; Fastrés, A.; Gommeren, K.; Saegerman, C.; Clercx, C. A questionnaire-based survey of owner-reported environment and care of West Highland white Terrier with or without idiopathic pulmonary fibrosis. In Proceedings of the 24th ECVIM-CA Congress, Mainz, Germany, 4-6 Sep. 2014.

What is claimed is:

1. A method for treating a subject for idiopathic pulmonary fibrosis (IPF), the method comprising the steps of:
extracting genomic DNA from a sample from the subject;
assaying the genomic DNA for one or more single nucleotide polymorphisms (SNPs);
detecting at least one of:
a T allele at position 54992254 on canine (CanFam3.1) chromosome 18,
a C allele at position 54987884 on canine (CanFam3.1) chromosome 18,
an A allele at position 54986491 on canine (CanFam3.1) chromosome 18,
an A allele at position 54986070 on canine (CanFam3.1) chromosome 18,
an A allele at position 54992285 on canine (CanFam3.1) chromosome 18,
a G allele at position 54987464 on canine (CanFam3.1) chromosome 18,
an A allele at position 54983627 on canine (CanFam3.1) chromosome 18,
a G allele at position 54984004 on canine (CanFam3.1) chromosome 18,
a C allele at position 54987912 on canine (CanFam3.1) chromosome 18, and
a C allele at position 54986170 on canine (CanFam3.1) chromosome 18; and
administering an effective amount of a treatment to the subject.

2. The method of claim 1, wherein the subject is a canine subject.

3. The method of claim 2, wherein the canine subject is selected from the group consisting of West Highland White Terrier, Scottish Terrier, and Bichons Frise.

4. The method of claim 1, wherein the treatment comprises administering an effective amount of a brohchodilator or a steroid.

5. The method of claim 1, wherein the treatment comprises administering an effective amount of an anti-fibrotic drug or an anti-inflammatory drug.

6. The method of claim 1, wherein the treatment comprises administering an effective amount of Nintedanib, Pirfenidone, prednisone, Mycophenolate mofetil, mycophenolic acid, Azathioprine, pamrevlumab, omeprazole, or cyclophosphamide.

7. The method of claim 1, wherein the treatment comprises oxygen therapy, pulmonary rehabilitation, or organ transplantation.

8. The method of claim 1, wherein assaying the genomic DNA comprises a method selected from the group consisting of whole genome sequencing, Sanger sequencing, next generation sequencing, pyrosequencing, sequencing by ligation, sequencing by synthesis, single molecule sequencing, pooled and barcoded DNA sequencing, PCR, real-time PCR, quantitative PCR, microarray analysis of genomic DNA, restriction fragment length polymorphism analysis, allele specific ligation, and comparative genome hybridization.

9. A method for diagnosing and treating a subject for idiopathic pulmonary fibrosis (IPF), the method comprising the steps of:
extracting genomic DNA from a sample derived from the subject;
determining in the subject-derived sample an expression of a gene selected from the group consisting of succinate dehydrogenase complex assembly factor 2 (SDHAF2), cleavage and polyadenylation specific factor 7 (CPSF7), and mucin 5B, oligomeric mucus/gel-forming (MUC5B);
diagnosing the subject as having IPF based on the expression of the gene being different than a normal control sample; and
administering to the subject diagnosed as having IPF an effective amount of a pharmaceutical composition selected from the group consisting of a brohchodilator, a steroid, an anti-fibrotic composition, and an anti-inflammatory composition.

10. The method of claim 9, wherein the pharmaceutical composition is selected from the group consisting of Nintedanib, Pirfenidone, prednisone, Mycophenolate mofetil, mycophenolic acid, Azathioprine, pamrevlumab, omeprazole, and cyclophosphamide.

11. The method of claim 9, wherein the subject is human.

12. The method of claim 9, wherein the subject is a canine subject.

13. The method of claim 12, wherein the canine subject is selected from the group consisting of West Highland White Terrier, Scottish Terrier, and Bichons Frise.

14. The method of claim 12, determining the expression of a gene further comprises detecting the SNPs corresponding to at least one of:
position 54992254 on canine (CanFam3.1) chromosome 18,
position 54987884 on canine (CanFam3.1) chromosome 18,
position 54986491 on canine (CanFam3.1) chromosome 18,
position 54986070 on canine (CanFam3.1) chromosome 18,
position 54992285 on canine (CanFam3.1) chromosome 18,
position 54987464 on canine (CanFam3.1) chromosome 18,
position 54983627 on canine (CanFam3.1) chromosome 18,
position 54984004 on canine (CanFam3.1) chromosome 18,
position 54987912 on canine (CanFam3.1) chromosome 18, and
position 54986170 on canine (CanFam3.1) chromosome 18.

15. The method of claim 14, further comprising diagnosing the subject as having IPF based on the expression of the gene being characterized by at least one of:
a T allele at position 54992254 on canine (CanFam3.1) chromosome 18,
a C allele at position 54987884 on canine (CanFam3.1) chromosome 18,
an A allele at position 54986491 on canine (CanFam3.1) chromosome 18,
an A allele at position 54986070 on canine (CanFam3.1) chromosome 18,
an A allele at position 54992285 on canine (CanFam3.1) chromosome 18,
a G allele at position 54987464 on canine (CanFam3.1) chromosome 18,
an A allele at position 54983627 on canine (CanFam3.1) chromosome 18,
a G allele at position 54984004 on canine (CanFam3.1) chromosome 18,
a C allele at position 54987912 on canine (CanFam3.1) chromosome 18, and
a C allele at position 54986170 on canine (CanFam3.1) chromosome 18;

16. A method for breeding a canine subject to reduce propensity to canine idiopathic pulmonary fibrosis (CIPF) in progeny resulting from the breeding, the method comprising the steps of:
extracting genomic DNA from a sample from the canine subject;
assaying the genomic DNA for one or more single nucleotide polymorphisms (SNPs);
detecting the SNPs corresponding to at least one of:
position 54992254 on canine (CanFam3.1) chromosome 18,
position 54987884 on canine (CanFam3.1) chromosome 18,
position 54986491 on canine (CanFam3.1) chromosome 18,
position 54986070 on canine (CanFam3.1) chromosome 18,
position 54992285 on canine (CanFam3.1) chromosome 18,
position 54987464 on canine (CanFam3.1) chromosome 18,
position 54983627 on canine (CanFam3.1) chromosome 18,
position 54984004 on canine (CanFam3.1) chromosome 18,
position 54987912 on canine (CanFam3.1) chromosome 18, and
position 54986170 on canine (CanFam3.1) chromosome 18; and
breeding the canine subject with at least one of:
an A allele at position 54992254 on canine (CanFam3.1) chromosome 18,
a T allele at position 54987884 on canine (CanFam3.1) chromosome 18,
a G allele at position 54986491 on canine (CanFam3.1) chromosome 18,
a G allele at position 54986070 on canine (CanFam3.1) chromosome 18,
a G allele at position 54992285 on canine (CanFam3.1) chromosome 18,
a T allele at position 54987464 on canine (CanFam3.1) chromosome 18,
a G allele at position 54983627 on canine (CanFam3.1) chromosome 18,
an A allele at position 54984004 on canine (CanFam3.1) chromosome 18,
a T allele at position 54987912 on canine (CanFam3.1) chromosome 18, and
a T allele at position 54986170 on canine (CanFam3.1) chromosome 18.
